(19) United States
(12) Patent Application Publication
(10) Pub. No.: US 2013/0280747 A1
(43) Pub. Date: Oct. 24, 2013

(54) CELL CULTURE APPARATUS

(71) Applicant: San Diego State University Research Foundation, San Diego, CA (US)

(72) Inventors: Karen Diane MAY-NEWMAN, San Diego, CA (US); Irwin Hai-Wen LING, Encinitas, CA (US); John Nicholas SAM-SOON, Moka (MU); York Kin WONG, San Diego, CA (US)

(21) Appl. No.: 13/790,455

(22) Filed: Mar. 8, 2013

Related U.S. Application Data
(60) Provisional application No. 61/608,528, filed on Mar. 8, 2012.

(51) Int. Cl. G01N 33/50

(52) U.S. Cl. CPC G01N 33/5005
USPC 435/29; 435/305.2

(57) ABSTRACT

The invention provides a unique cell culture chamber, as well as systems for culturing and analyzing cells, methods of using the chamber, and techniques for culturing and analyzing cells. The invention also provides an apparatus for distributing cells across a substrate, comprising a chamber comprised of at least one polydimethylsiloxane (PDMS) surface, where the chamber comprises one or more wells, and each well can contain a volume of a suspension of cells in culture, and where the chamber, when placed over a substrate, provides a more uniform dispersement of cells on the substrate than when the cells are deposited on a substrate without said chamber.

### CROSS-REFERENCE TO RELATED APPLICATIONS

This application claims the benefit of U.S. provisional application No. 61/608,528, filed on 8 Mar. 2012, and which application is incorporated herein by reference.

### BACKGROUND OF THE INVENTION

Three factors have been shown to play major roles in determining the fate of a population of cells, in particular the differentiation of stem cells: cell-cell interactions, cell-extracellular matrix interactions, and the presence or absence of specific growth factors. The difficulty has been to examine the effects of one of these factors in the absence of the other two. A platform has been developed commercially by MicroStem, Inc., that is a hydrogel-coated microscope slide spotted with up to 1,000 different combinations of ECM proteins. Cells are applied to the screening slide by first suspending them in media then adding them to the surface of the slide.

During the cell attachment period there are two competing reactions ongoing: the tendency of the cells to adhere to each other (aggregate), and the recognition by the cells of the proteins that are on the slide. Both of these activities will affect the fate of the cells and it would be beneficial to suppress the first effect long enough for the cells to recognize the proteins attached to the slide.

What has been found previously is that approximately 10-20 cells attach to each spot. Large aggregates of cells can be observed under the microscope that are still in suspension and do not attach to any of the spots. There is also a high variability in the occupancy of different regions on the slide. Generally the spots in the center will have a higher number of cells attached to them while those on the periphery have fewer or sometimes no cells attached. The result is that only about 4-8% of the initially applied cells attach to the slide and there is a high spot-to-spot variability in the average numbers of cells/spot. These data also imply that the vast majority of the cells never attach and are discarded as aggregates in suspension. Since stem cells are difficult to obtain from most tissues, throwing away over 90% of them severely limits the number of experiments that can be attempted with each batch of cells.

Accordingly, there is a need for cell culture chambers and techniques that reduce aggregation of cells, increase the adherence of cells to a surface, and reduce the amount of cells that are washed from a surface prior to analysis.

### SUMMARY

The invention provides a novel cell culture apparatus and methods for using the apparatus. The invention provides methods for producing increased homogeneous distributions of cells over a culture surface. While the improvement in cell loss was significant, the effort to more uniformly distribute the suspended cell solution was affected by the surface coating. Our studies found that the cell aggregation observed when culturing the cells over the hydrogel-coated slides was produced by the hydrogel coating, which was unexpected. The hydrogel coating is designed to repel the cells and minimize cell adhesion outside the spots of deposited proteins. Uncoated slides may benefit only from having the cell loss reduced.

Accordingly, the invention provides a cell culture apparatus comprising a cell culture chamber, the chamber comprising a polydimethylsiloxane surface, wherein the polydimethylsiloxane surface reduces cell aggregation on a substrate, provides increased cell adhesion to a substrate, and affords a more uniform dispersement of cells across a substrate.

The invention also provides methods to increase the number of adhered cells and/or increase the adherence of cells to a surface for analysis of the cells comprising carrying out the cell application and washing techniques described herein. The invention further provides methods to reduce intracellular adhesion comprising carrying out the cell application and washing techniques described herein. The invention further provides methods to disperse cells throughout a cell culture media comprising adding cells to a cell culture media and apparatus as described herein, thereby dispersing the cells in a substantially uniform distribution across the cell culture media.

The entire cell culture apparatus can be made of polydimethylsiloxane (PDMS), and the cells can be any cells that can be analyzed in a laboratory setting, including, but not limited to, immortal cells, cancer cells, stem cells, mammalian cells, plant cells, fungal cells, bacterial cells, transformed cells, primary cells, eukaryotic cells, prokaryotic cells, or any other cell type, or any cells derived from cells of any type.

The invention provides an apparatus for distributing cells across a surface, comprising a chamber comprised of at least one polydimethylsiloxane (PDMS) surface, wherein the chamber comprises one or more depressions, wherein the depressions can contain a volume of a suspension of cells in culture, and wherein the chamber, when placed over a substrate, provides a more uniform dispersement of cells on the substrate than when the cells are deposited on a substrate without said chamber.

Optionally, the polydimethylsiloxane surface is attached to the substrate. In another embodiment, the chamber further provides a reduction in cell aggregation on the substrate as compared to the cell aggregation on a substrate without said chamber. Additionally, the chamber of the apparatus provides increased cell adherence to the substrate as compared to the cell adherence on a substrate without said chamber. In a further embodiment, the chamber is comprised of up to 200 depressions or wells. Optionally, the chamber of the apparatus has variable permeability to gas and solution, and wherein hypoxic or normoxic conditions can be induced inside the chamber.

In certain embodiments of the invention, the substrate is a microscope slide. In an embodiment, the substrate used with the apparatus is a slide, optionally coated with a hydrogel, an extracellular matrix protein, or a bioactive molecule, or a combination thereof.

In another embodiment, a cell culture apparatus is provided, which comprises a polydimethylsiloxane chamber and a substrate, wherein polydimethylsiloxane chamber attaches to the substrate, and wherein the polydimethylsiloxane surface 1) reduces cell aggregation on the substrate, 2) increases cell adhesion to the substrate, 3) provides a more uniform dispersement of cells across the substrate, or a combination thereof, as compared to a cell culture apparatus not containing a polydimethylsiloxane chamber.

Further provided by the invention is a method for the uniform distribution of cells across a substrate, comprising 1) attaching a PDMS chamber to a substrate, 2) placing a volume of cells into the PDMS chamber, 3) allowing the cells to adhere to the substrate, wherein the chamber provides a uniform dispersement of cells on the substrate. The method of the invention optionally comprises removing the PDMS chamber from the substrate after the cells adhere to the substrate.

In one embodiment of the methods of the invention, the chamber is the chamber provided in the apparatus described herein. In an embodiment of the methods of the invention, the chamber does not affect cell viability. In a further embodiment of the methods of the invention, the use of the chamber provides for a reduction in the quantity of cells required to seed the substrate, as compared to the quantity of cells required to seed the substrate without the use of the chamber. In other embodiments of the methods of the invention, the use of the chamber provides increased cell adherence to the substrate as compared to the cell adherence on a substrate without said chamber. The methods described herein optionally include treating the chamber with a bioactive substance to induce or inhibit cell functions, prior to the attachment of the chamber to the substrate. The methods described herein optionally include treating the chamber to render the PDMS hydrophilic or hydrophobic, prior to the attachment of the chamber to the substrate. The methods provided by the invention optionally include a substrate that is coated with a hydrogel, an extracellular matrix protein, or a bioactive molecule, or a combination thereof. In the apparatus and the methods provided herein, the cells can be stem cells, rare cells, mammalian cells, animal cells, plant cells, or fungal cells.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention. FIGS. 1-9 show particular embodiments of the invention; however the invention is not limited to such embodiments.

FIG. 1. Diagram of MicroMatrix™ 36 system by Microstem Inc.

FIG. 2. 24 hrs after static cell culture A) Cell aggregation at the center of slide B) Low cell population at peripheries.

FIG. 3. Photographs of the PDMS prototypes: A) 36 individual chamber design and B) mono-well design.

FIG. 4. A) MicroMatrix™ 36 slide during cell culturing. The grid is arranged in 3 columns (labeled 1-3) and 12 rows (A-L). B) Schematic of illustration of the PDMS chamber to a slide.

FIG. 5. Photographs of cell deposition in the microwells at peripheral locations, after washing: A) using the PDMS chamber, and B) using the standard or traditional approach.

FIG. 6. Contour plots of change in cell count ratio at each of the 36 microwell locations, A) before washing, and B) after washing. Blue color indicates an increase in cell count using the PDMS method relative to the control method, while red color indicates a decrease in cell count.

FIG. 7. Table of average cell counts per spot for PDMS method (left panel) and standard or traditional method (right panel).

FIG. 8. Image Analysis Flowchart.

FIG. 9. Uniform CHO cells distribution A) along the edge and B) in the center of a non-hydrogel-coated slide, but non-uniform distribution C) along the edge and D) in the center of a hydrogel-coated (without ECMP present).

FIG. 10. Photograph of the PDMS chamber attached to a slide, showing the reduced volume of cells needed for the cell culture apparatus and methods of the invention.

FIG. 11. Photograph of the PDMS chamber attached to a slide, showing the reduced volume of cells needed for the cell culture apparatus and methods of the invention.

FIG. 12. Photograph of the PDMS chamber being placed on a standard microscope slide.

FIG. 13. Photograph of the PDMS chamber in use with a Microstem™ slide.

### DETAILED DESCRIPTION

A polydimethylsiloxane (PDMS) culture chamber was developed to confine cells to a small volume over the spotted area of the MicroStem slide during the culturing process. The culture chamber has depressions or wells to hold suspended cells while they attach to a surface. Cells are pipetted onto the PDMS chamber wells, the desired culture surface is attached, and the entire assembly is incubated until the cells attach to the surface. Alternately, the PDMS chamber and the substrate or culture surface are attached, the cells are pipetted into the PDMS chamber wells, and the entire assembly is incubated until the cells attach to the surface. Following cell adhesion, the culture chamber can be removed and discarded.

PDMS is an ideal material due to its high optical clarity, flexibility, biocompatibility, and high gas permeability which allows the small volume design to minimize cell stress and maximize cell viability. A major advantages of this chamber is that it reduces the number of cells needed for culturing, which is important when using rare cells types such as stem cells, and it reduces the nonhomogeneous distribution of cells across the culture surface, which enables a more consistent cell culture and reduces regional variability, including spot-to-spot differences. PDMS is a biocompatible substrate for cell culture. See, for example, Lee et al., Langmuir, 20, 11684-11691 (2004). A substrate such as PDMS is useful for cell culture due to its transparency, as well as its oxygen permeability. Bacteria and viruses do not pass through PDMS membranes, making a PDMS chamber useful for reducing contamination in cell cultures and equipment. PDMS can be rendered hydrophobic or hydrophilic, depending on need.

The process of culturing cells involves depositing or “plating” a solution of suspended cells on top of a substrate such as a coated or uncoated glass slide. The suspension is often applied with a pipette and time allowed for cells to settle to the surface and attach. On some surfaces, the cells do not become uniformly distributed but aggregate near the center or at the edges of the dish. This lack of uniformity hampers applications such as screening that rely on an equal probability for cells to attach anywhere on the surface. A soft flexible silicone cell culture chamber with one or more wells was developed that attaches to the top of a microscope slide to confine and distribute a solution of suspended cells in contact with the slide surface. The culture chamber greatly reduces the volume of cells needed as well as reducing the variation in cell distribution.

In one embodiment, the PDMS cell culture chamber provides the following components and properties. A current embodiment includes a microscope slide and suspended cells in solution during the incubation and attachment process (24 h). The apparatus (slide) can have one monowell or depression or up to 200 small wells or depressions. The apparatus can have variable permeability to gas and solution, which can be used to induce hypoxic or normoxic conditions inside the chamber. The apparatus can be coated with bioactive substances to induce or inhibit cell functions. The apparatus can be treated to render PDMS hydrophilic or hydrophobic.

In another embodiment, the PDMS cell culture chamber allows pathogenic cells in culture to be contained or confined within the chamber. This unique feature allows potential or known pathogenic cell cultures to be incubated or evaluated in close proximity to non-pathogen cell cultures, which provides a great savings in cost as well as a great increase in safety to the researcher and the environment, without fear of contaminating the non-pathogenic cell cultures.

The PDMS chamber provided herein fits over a substrate. Substrates compatible for the PDMS chamber described herein include but are not limited to glass slides, slides coated with a hydrogel, slides containing ECM or bioactive molecules on their surfaces, where the ECM or bioactive molecules may be patterned on the slide surface or not patterned on the slide surface. Additional substrates compatible for the PDMS chamber described herein also include cell culture dishes or plates or multi-well plates, made of standard materials or a custom material, where the surfaces of the dishes or plates are coated with a hydrogel, or are coated with ECM or bioactive molecules on their surfaces, where the ECM or bioactive molecules may be patterned on the dish or plate surface or not patterned on the dish or plate surface.

### DEFINITIONS

As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with the recitation of claim elements or use of a “negative” limitation.

The term “and/or” means any one of the items, any combination of the items, or all of the items with which this term is associated. The phrase “one or more” is readily understood by one of skill in the art, particularly when read in context of its usage. For example, one or more components in a mixture can refer to one, one or two, one to about three, one to about four, or one to five, depending on the context of the usage.

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percents, proximate to the recited range that are equivalent in terms of the functionality of the individual ingredient, the composition, or the embodiment.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percents or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, as used in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to provide a desired effect.

As used herein, “ECM” is an abbreviation for the phrase “extracellular matrix”. ECM is composed of proteins and polysaccharides. Connective tissue is largely ECM together with a few cells. For cells ECM provides mechanical support, a biochemical barrier, a medium for the extracellular communication that is assisted by CAMs (cell adhesion molecules), the stable positioning of cells in tissues through cell matrix adhesion, and the repositioning of cells by cell migration during cell development and wound repair. ECM proteins are macromolecular organic compounds that contain carbon, hydrogen, oxygen, nitrogen, and usually, sulfur. These macromolecules (proteins) form an intricate meshwork in which cells are embedded to construct tissues. Variations in the relative types of macromolecules and their organization determine the type of extracellular matrix, each adapted to the functional requirements of the tissue. The two main classes of macromolecules that form the extracellular matrix are: glycosaminoglycans, usually linked to proteins (proteoglycans), and fibrous proteins.

Cell culture, as referred to herein, is a means to artificially cultivate cells in a laboratory or production-scale device (i.e., in vitro). The cells can be cultured in either a batch or continuous process device.

The cells may be cells capable of culture or artificial cultivation, including but not limited to, immortal cells, stem cells, rare cells, mammalian cells, bacterial cells, prokaryotic cells, fungal cells, plant cells, animal cells, bone marrow stem cells, primary cells, epithelial cells, hepatic cells, fibroblasts, cancer cells, eukaryotic cells, prokaryotic cells, transformed or genetically altered cells of any type or origin, any cells derived from cells of any type and any other cells capable of culture or artificial cultivation.

In certain embodiments, the methods of the invention are for use with static cultures. In other embodiments, the methods of the invention can be used with cells cultured under fluidic or microfluidic conditions. See, for example, the cells types discussed in Kim, et al., Lab Chip, (7), 681-694 (2007).

The standard or traditional approach for plating cells involves pipetting a volume of cells into each well of a plate or petri dish, or other vessel used for this purpose, or slide. For plating onto a Microstem slide, see http://www.microstem.com/sites/default/files/details/MicroMatrix %2036%20Directions %20For %20Use_{—}0.pdf.

The surfaces or slides that can be used with the chamber of the invention include, but are not limited to, slides that are coated with hydrogels or other coatings, including those compatible with cell culture techniques, slides that are not coated, or any other suitable vessel or surface.

The PDMS chamber may be of any shape desired. In one embodiment, the PDMS chamber can be rectangular, as shown in the figures, and can fit over a standard size microscope slide. In another embodiment, the PDMS chamber can be circular, so as to fit over the bottom of a standard or circular shaped Petri dish. In a further embodiment, the PDMS chamber can be square, and therefore able to fit over a standard size cover slip on a Petri dish or microscope slide, or other surface. In a further embodiment, the PDMS chamber can be fashioned to fit over a multi-well cell culture plate, or a 96 well plate, thereby allowing a more homogeneous cell distribution within each well.

### TECHNICAL DETAILS

The stamp can be prepared by (1) creating a positive mold out of PC-ABS using a rapid prototyping printer, (2) making a negative mold out of PDMS on one side of the positive mold and (3) bombarding the PDMS surface with oxygen radicals to create a hydrophilic surface.

If a hydrophilic chamber surface is desired, the chamber can undergo oxygen plasma surface modification, according to protocols known in the art. As a non-limiting example, the chamber can subjected to oxygen plasma treatment for between 1-60 seconds or between 5-10 seconds, at between 50-100 watts or between 60-80 watts, at between 50-1000 mTorr. As another non-limiting example, the chamber can subjected to oxygen plasma treatment for approximately 5-10 seconds, at between 60-80 watts, and at between 300-600 mTorr.

Working prototypes of the cell chamber have been developed and tested several times with CHO (Chinese Hamster Ovary) cells with great success. A recent prototype uses a monowell design and demonstrated an average difference in cell density between center-to-corner of 4%, center-to-short edge of 24%, and center-to-long edge of 27%. This contrasted with traditional plating methods where the cell density difference averaged 90% difference between center-to-edges.

The following Examples are intended to illustrate the above invention and should not be construed as to narrow its scope. One skilled in the art will readily recognize that the Examples suggest many other ways in which the invention could be practiced. It should be understood that numerous variations and modifications may be made while remaining within the scope of the invention.

### EXAMPLES

### Example 1

### Evaluation of In-Vitro Screening Platform

Stem cells have an important role to play in the field of regenerative medicine because of their ability to differentiate into any cell type in the body. However, numerous inputs such as growth factors, cell-cell interactions, and extracellular matrix (ECM) are required for properly regulated differentiation. This in turn renders in vitro studies laborious and difficult due to the sheer amount of components that must be regulated.

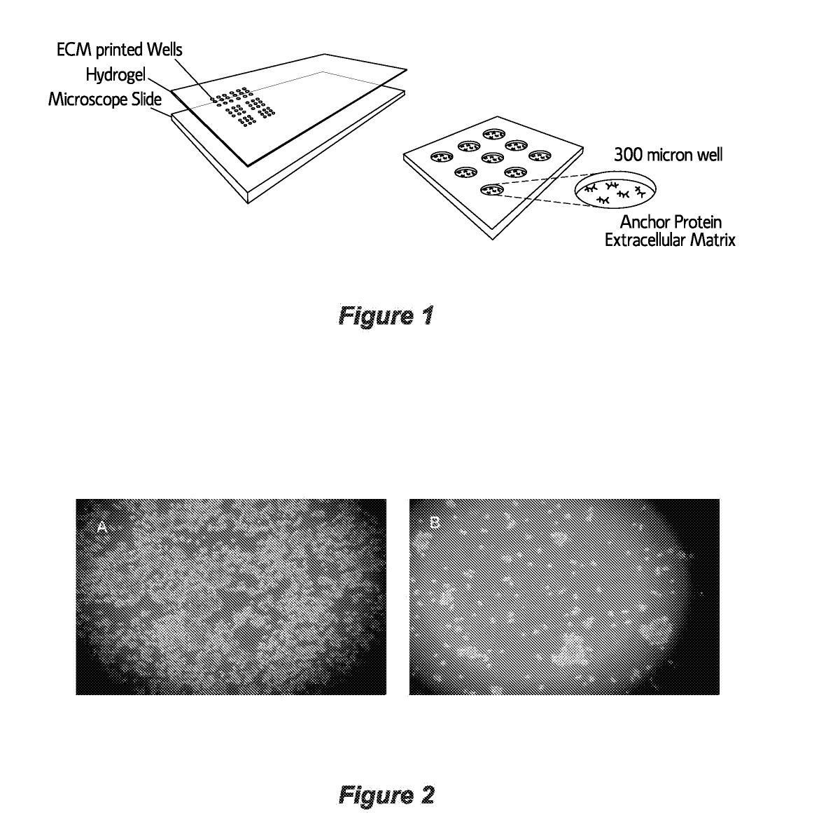

MicroStem, Inc. (California, U.S.A.; http://www.microstem.com) has developed an in-vitro screening platform that includes a 75×25 mm hydrogel-coated microscope slide on which ECM protein spots have been microprinted (MicroMatrix™ 36 ECM Array) (FIG. 1). Cells will exclusively attach to these ECM spots, and be repelled by the hydrogel. Stem cells can be seeded onto the slide, and the influence from the protein spot combination on cell growth and differentiation assessed using fluorescent stains.

However, this screening platform is subject to cellular aggregation particularly in the center of the culture plate (FIG. 2). This causes unequal numbers of cells to settle in and around each microwell, which compromises statistical studies in the screening process. Furthermore, since each microwell can only accommodate a relatively small number of cells, a large number of cells used during seeding are un-used and discarded (˜90% loss).

### Example 2

### Reduction of Intercellular Adhesion Using a Novel Cell Culture System

Techniques and apparatuses were developed to overcome the difficulties in using available screening technology. Described herein below are apparatuses and techniques for:
Reduced cell aggregation during culturing and settling.
Minimized cell loss during the seeding process.
Maximized cell viability during the seeding process.
Ensuring that during the seeding process, each microwell has an equal opportunity of receiving the same number of cells.

### Methods.

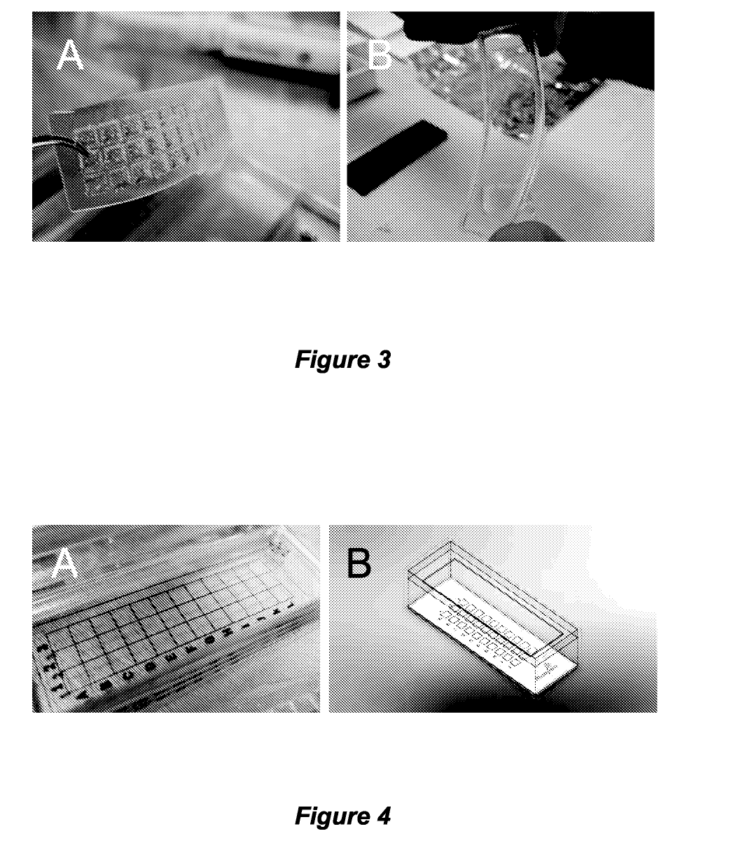

A PDMS (polydimethylsiloxane) based design (FIG. 3) was selected to confine cells in a small volume, while maintaining gas permeability and biocompatibility. Each PDMS chamber contains 0.6-1.0 ml of CHO (Chinese Hamster Ovary) cells in F-12K media at 1×10⁵ cells/ml.
The PDMS chamber was configured to easily align with the microwell array grid and applied to the MicroMatrix slide (FIG. 4). In other applications, the PDMS chamber can be configured to align with any desired array grids or patterns, and used with any appropriate or desired type of slide.

Molds and PDMS Chamber Fabrication.

The PC-ABS (Polycarbonate/Acrylonitrile Butadiene Styrene) master molds can be made from any suitable fabrication processes known to the art. In certain embodiment, the PC-ABS master mold was created using a 3D rapid prototyping machine (Fortus 400mc), while the acrylic molds were CNC machined (Haas VF-2YT). Liquid PDMS is poured onto the master mold, de-gassed, and oven cured until target stiffness is achieved. The chamber fabrication procedure is as follows: PDMS resin is mixed with a curing agent at 10:1 wt, the mixture is centrifuged (which serves to degas and debubble the mixture) and subjected to vacuum degassing, the mixture is then poured into the appropriate form or container and baked at 60° C. for approximately 4 hours. Various centrifugation speeds can be used, depending on the material used and the desired outcome. In certain embodiments, a speed of between approximately 1500-4000 rpms can be used, and in other embodiments, a speed of between approximately 2500-3200 rpms can be used, and in still other embodiments, a speed of between approximately 2800-3000 rpms can be used. The speed of the centrifuge must not be such that any detrimental effects occur to the PDMS mixture. The removal of the bubbles was verified visually; however, it can be done in any appropriate manner. Following baking, the PDMS is removed from the mold and excess material is trimmed. Alterations and adjustments may be made to the PDMS chamber protocol, depending on desired chamber characteristics.

In certain embodiments of the invention, when a hydrophilic surface is desired, the oxygen plasma surface modification treatment can be done at 70 watts, at 500 mTorr, for 10 seconds.

### PDMS Chamber Testing:

Testing platforms used: 1) standard MicroMatrix36 slides from MicroStem (FIG. 4), 2) slides with hydrogel but no ECMPs, and 3) glass slides without hydrogel or ECMPs;
PDMS+slide assemblies;
Control seeding with 5 ml of CHO cells at 5×10⁴ cells/ml;
Culture for 12-24 hours; non-adhered cells are washed away by PBS solution.

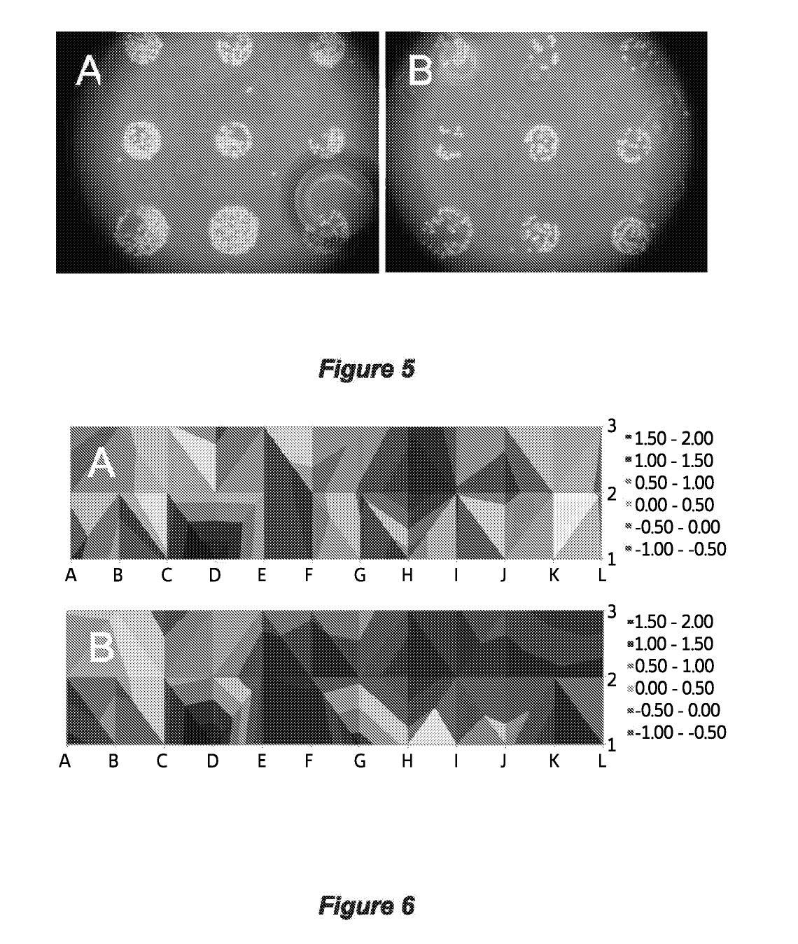

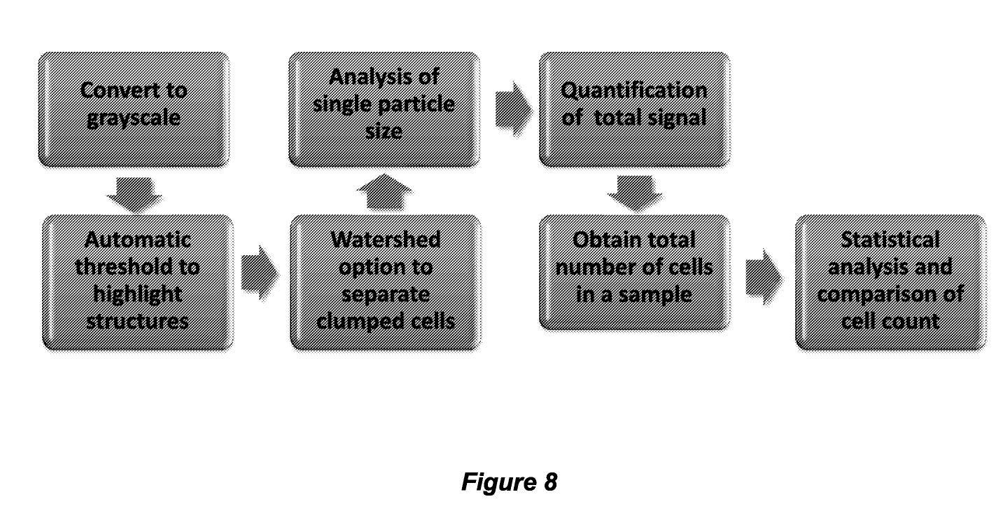

Results of the analysis are shown in FIGS. 5-7 and an image analysis flowchart is shown in FIG. 8.

### Microscopy & Cell Distribution Analysis

Cells were observed and photographed 12-24 hrs after seeding by phase contrast microscope.
The images were analyzed with ImageJ (NIH) and quantification performed following the automatic cell counting algorithm flowchart (FIG. 8).
To verify seeding distribution, cells still in suspension and attached cells were counted prior to washing.
Only attached cells are counted after washing to analyze attachment efficiency.

### Results

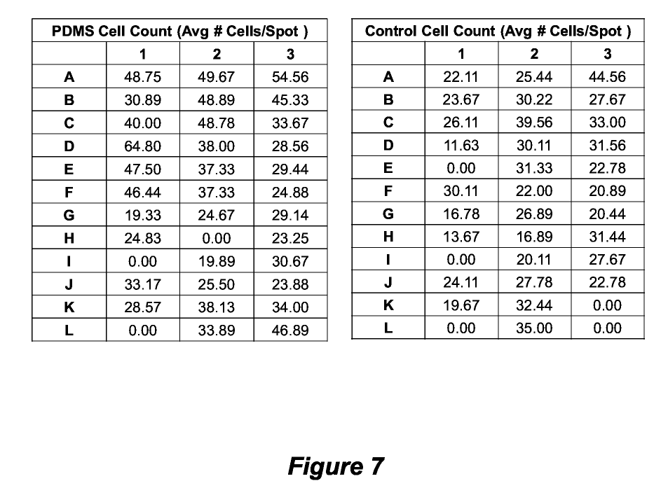

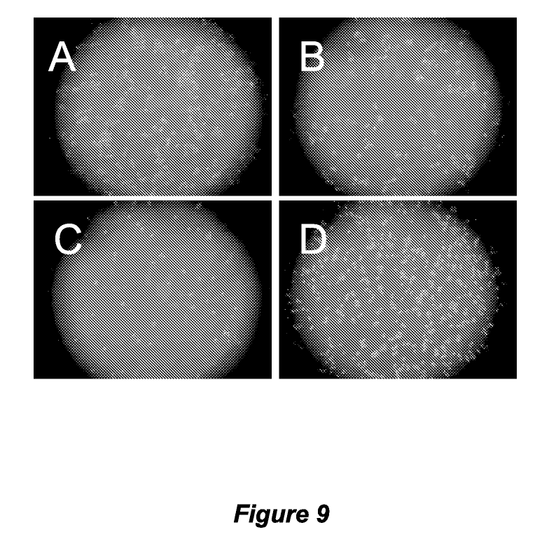

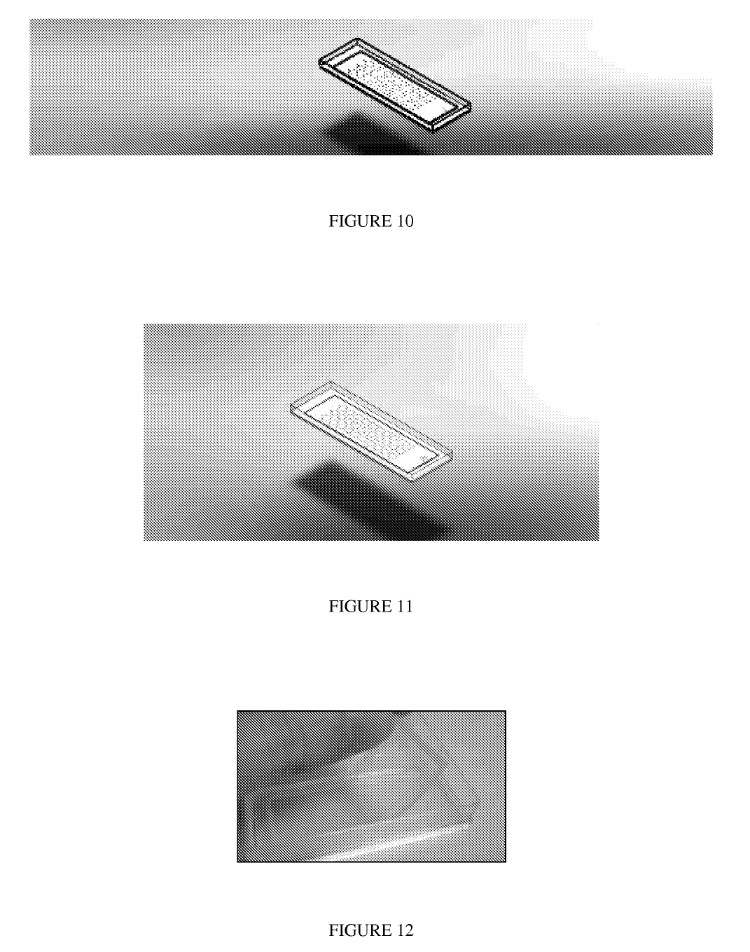

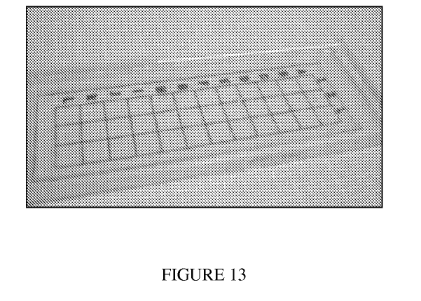

We previously determined that the MicroMatrix hydrogel layer was a significant contribution to the non-uniformity of the cell aggregation (see Example 1) (FIG. 9). By using a PDMS chamber, the cells are confined from bulk fluid movement, and the chamber counter-balances the repulsion forces from the hydrogel. These results indicate an overall increase in cells prior to PDMS removal and washing (FIGS. 6A and 7), and a decrease in excess cells along the center of the slide (FIG. 6B). Successful culturing 24-hours after PDMS chamber removal shows that cell viability is maintained. The PDMS chamber design provides a convenient method to distribute cells uniformly across slides, including hydrogel slides, while reducing the overall quantity of cells required for seeding, without substantially impacting cell viability.

Useful information and techniques are described in the following publications:
[1] J C McDonald, & G M Whitesides. “Poly(dimethylsiloxane) as a Material for Fabricating Microfluidic Devices.” Acc. Of Chem. Research. 25 (2002): 491-499.
[2] S W Rhee, et al. “Patterned Cell Culture inside Microfluidic Devices”. Lab on a Chip. 5 (2005): 102-107.
[3] N Li, A Tourovskaia, & A Folch. “Biology on a Chip: Microfabrication for Studying the Behavior of Cultured Cells.” Critical Revs. In Biomed. Eng. 31 (2003): 423-488

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference. No limitations inconsistent with this disclosure are to be understood therefrom. The invention has been described with reference to various specific and preferred embodiments and techniques. However, it should be understood that many variations and modifications may be made while remaining within the spirit and scope of the invention.

We claim:

1. An apparatus for distributing cells across a surface, comprising a chamber comprised of at least one polydimethylsiloxane (PDMS) surface, wherein the chamber comprises one or more depressions, wherein the depressions can contain a volume of a suspension of cells in culture, and wherein the chamber, when placed over a substrate, provides a more uniform dispersement of cells on the substrate than when the cells are deposited on a substrate without said chamber.

2. The apparatus of claim 1, wherein the polydimethylsiloxane surface is attached to the substrate.

3. The apparatus of claim 1, wherein the substrate is a slide.

4. The apparatus of claim 1, wherein the substrate is coated with a hydrogel, an extracellular matrix protein, or a bioactive molecule, or a combination thereof.

5. The apparatus of claim 1, wherein the chamber further provides a reduction in cell aggregation on the substrate as compared to the cell aggregation on a substrate without said chamber.

6. The apparatus of claim 1, wherein the chamber provides increased cell adherence to the substrate as compared to the cell adherence on a substrate without said chamber.

7. The apparatus of claim 1, wherein the cells are stem cells, rare cells, mammalian cells, animal cells, plant cells or fungal cells.

8. The apparatus of claim 1, wherein the chamber is comprised of up to 200 depressions.

9. The apparatus of claim 1, wherein the chamber has variable permeability to gas and solution, and wherein hypoxic or normoxic conditions can be induced inside the chamber.

10. A method for the uniform distribution of cells across a substrate, comprising,
1) attaching a PDMS chamber to a substrate, 2) placing a volume of cells into the PDMS chamber, 3) allowing the cells to adhere to the substrate, wherein the chamber provides a uniform dispersement of cells on the substrate.

11. The method of claim 10, wherein the chamber is the chamber provided in claim 1.

12. The method of claim 10, further comprising removing the PDMS chamber from the substrate after the cells adhere to the substrate.

13. The method of claim 10, wherein the chamber does not affect cell viability.

14. The method of claim 10, wherein the use of the chamber provides for a reduction in the quantity of cells required to seed the substrate, as compared to the quantity of cells required to seed the substrate without the use of the chamber.

15. The method of claim 10, wherein the chamber provides increased cell adherence to the substrate as compared to the cell adherence on a substrate without said chamber.

16. The method of claim 10, wherein prior to the attachment of the chamber to the substrate, the chamber is treated with a bioactive substance to induce or inhibit cell functions.

17. The method of claim 10, wherein prior to the attachment of the chamber to the substrate, wherein the chamber is treated to render the polydimethylsiloxane hydrophilic or hydrophobic.

18. The method of claim 10, wherein the cells are stem cells, rare cells, mammalian cells, animal cells, plant cells, or fungal cells.

19. The method of claim 10, wherein the substrate is coated with a hydrogel, an extracellular matrix protein, or a bioactive molecule, or a combination thereof.

20. A cell culture apparatus, comprising a polydimethylsiloxane chamber and a substrate, wherein polydimethylsiloxane chamber attaches to the substrate, and wherein the polydimethylsiloxane surface 1) reduces cell aggregation on the substrate, 2) increases cell adhesion to the substrate, 3) provides a more uniform dispersement of cells across the substrate, or a combination thereof, as compared to a cell culture apparatus not containing a polydimethylsiloxane chamber.
